(19) United States
(12) Patent Application Publication
(10) Pub. No.: US 2024/0390140 A1
(43) Pub. Date: Nov. 28, 2024

(54) GUIDE CATHETER FOR AN IMPLANT DELIVERY APPARATUS

(71) Applicant: EDWARDS LIFESCIENCES CORPORATION, Irvine, CA (US)

(72) Inventors: Kurt Kelly Reed, Costa Mesa, CA (US); Kevin Jiang, Irvine, CA (US); Alyssa Joy Hamm (formerly Gross), Huntington Beach, CA (US)

(21) Appl. No.: 18/798,230

(22) Filed: Aug. 8, 2024

Related U.S. Application Data
(63) Continuation of application No. PCT/US2023/013505, filed on Feb. 21, 2023.
(60) Provisional application No. 63/268,322, filed on Feb. 22, 2022.

(51) Int. Cl. A61F 2/24

(52) U.S. Cl. CPC A61F 2/2433; A61F 2/2436

(57) ABSTRACT

Devices and methods for providing fluid to a lumen of a guide catheter with a fluid reservoir fluidly coupled to the lumen, such that pressure within the lumen is maintained at a desired level, are disclosed. As one example, a delivery apparatus can include a handle, a shaft extending within and distally from the handle and having a main lumen, and a reservoir fluidly coupled to the main lumen. The reservoir is filled with fluid and has an adjustable fluid volume, and the reservoir is configured to passively supply fluid to the main lumen based on a fluid pressure in the main lumen.

### CROSS-REFERENCE TO RELATED APPLICATIONS

This application is a continuation of PCT patent application no. PCT/US2023/013505 filed on Feb. 21, 2023, which application claims the benefit of U.S. Provisional Patent Application No. 63/268,322, filed Feb. 22, 2022, each of which application being incorporated herein in its entirety by this specific reference.

### FIELD

The present disclosure relates to guide catheters for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing an implant delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The implant delivery apparatus can be inserted through a lumen of the guide catheter to help direct the implant delivery apparatus to a target implantation site (e.g., a native valve region) within the patient and/or help position the implant delivery apparatus at the target implantation site.

### SUMMARY

Described herein are prosthetic heart valves, docking devices, delivery apparatuses, guide catheters, and methods for implanting docking devices and prosthetic heart valves. The disclosed guide catheters can, for example, be configured to receive a portion of a delivery apparatus within a main lumen of the guide catheter in order to introduce the delivery apparatus into a patient's vasculature and guide the delivery apparatus toward a target implantation site for a prosthetic medical device mounted on the delivery apparatus. In some examples, the guide catheter can include a reservoir disposed within or external to a handle of the guide catheter and configured to passively supply fluid to a main lumen of the guide catheter such that a volume and pressure within the main lumen is maintained. As such, the devices and methods disclosed herein can, among other things, overcome one or more of the deficiencies of typical guide catheters.

A delivery apparatus can comprise a handle and one or more shafts coupled to the handle.

In some examples, the handle can comprise a housing and a compressible reservoir disposed within the housing.

In some examples, the handle can comprise a housing and a reservoir disposed external to the housing.

In some examples, the reservoir can be filled with fluid and have an adjustable fluid volume.

In some examples, the shaft can extend within and distally from the handle and have a main lumen, where the main lumen is fluidly coupled to the reservoir by a first channel disposed within the handle.

In some examples, the one or more shafts can include a shaft extending within and distally from the handle and having a main lumen and the delivery apparatus comprises a bladder containing a fluid volume that is fluidly coupled to the main lumen.

In some examples, the bladder can comprise a flexible wall that is configured to conform to the fluid volume and contract inward as the fluid volume decreases.

In some examples, a delivery apparatus comprises a handle, the handle comprising a housing and a compressible reservoir disposed within the housing. The reservoir is filled with fluid and has an adjustable fluid volume. The delivery apparatus further comprises a shaft extending within and distally from the handle and having a main lumen, where the main lumen is fluidly coupled to the reservoir by a first channel disposed within the handle.

In some examples, a delivery apparatus comprises a handle, a shaft extending within and distally from the handle and having a main lumen, and a bladder containing a fluid volume that is fluidly coupled to the main lumen. The bladder comprises a flexible wall that is configured to conform to the fluid volume and contract inward as the fluid volume decreases.

In some examples, a delivery apparatus comprises a handle, a shaft extending within and distally from the handle and having a main lumen, and a reservoir fluidly coupled to the main lumen, wherein the reservoir is filled with fluid and has an adjustable fluid volume, and wherein the reservoir is configured to passively supply fluid to the main lumen based on a fluid pressure in the main lumen.

In some examples, a delivery apparatus comprises one or more of the components recited in Examples 1-12, 34-42, and 57-73 below.

A delivery assembly can comprise an implant catheter and a guide catheter.

In some examples, the guide catheter can comprise a handle and a shaft extending distally from within the handle, the shaft having a main lumen that is configured to receive a portion of the implant catheter therethrough.

In some examples, the handle can comprise a housing and a flush port coupled to the housing.

In some examples, the guide catheter can comprise a compressible reservoir disposed within the housing, where the reservoir is filled with fluid and has an adjustable fluid volume, and where the flush port is fluidly coupled to the reservoir.

In some examples, the main lumen of the shaft can be fluidly coupled to the reservoir.

In some examples, the reservoir can be disposed around and radially outward of the shaft, and the main lumen can be fluidly coupled to the reservoir by a first channel extending between the main lumen and the reservoir.

In some examples, a delivery assembly comprises an implant catheter and a guide catheter. The guide catheter comprises a handle, the handle comprising a housing and a flush port coupled to the housing. The guide catheter further comprises a compressible reservoir disposed within the housing, where the reservoir is filled with fluid and has an adjustable fluid volume, and where the flush port is fluidly coupled to the reservoir. The guide catheter further comprises a shaft extending distally from within the handle and having a main lumen that is configured to receive a portion of the implant catheter therethrough, where the main lumen is fluidly coupled to the reservoir.

In some examples, a delivery assembly comprises one or more of the components recited in Examples 13-23 below.

A guide sheath can comprise a handle and a shaft extending within and distally from the handle.

In some examples, the handle can comprise a housing and a flush port coupled to the housing, and a seal housing assembly including one or more fluid seals.

In some examples, the handle can comprise a compressible reservoir disposed within the housing, distal to the seal housing assembly.

In some examples, the reservoir can be filled with fluid and have an adjustable volume and the flush port can be fluidly coupled to the reservoir.

In some examples, the shaft can have a main lumen that extends within the housing and through the seal housing assembly, the main lumen fluidly coupled to the reservoir.

In some examples, a guide sheath comprises a handle, the handle comprising a housing and a flush port coupled to the housing, a seal housing assembly including one or more fluid seals, and a compressible reservoir disposed within the housing, distal to the seal housing assembly. The reservoir is filled with fluid and has an adjustable volume and the flush port is fluidly coupled to the reservoir. The guide sheath further comprises a shaft extending within and distally from the handle and having a main lumen that extends within the housing and through the seal housing assembly, where the main lumen is fluidly coupled to the reservoir.

In some examples, a guide sheath comprises a handle comprising a housing and a flush port coupled to the housing. The guide sheath further comprises a reservoir fluidly coupled to the flush port and disposed external to the housing, wherein the reservoir is filled with fluid and has an adjustable volume. The guide sheath further comprises a shaft extending within and distally from the handle and having a main lumen that extends within the housing, wherein the main lumen is fluidly coupled to the reservoir by the flush port, and wherein the reservoir is configured to passively supply fluid to the main lumen based on a fluid pressure in the main lumen.

In some examples, a guide sheath comprises one or more of the components recited in Examples 24-33 and 74-82 below.

A method can comprise inserting a shaft of a guide catheter into a vessel of a patient and inserting a distal end portion of an implant catheter into a proximal end of the guide catheter and advancing the distal end portion of the implant catheter through the guide catheter toward a target implantation site for a prosthetic medical device mounted on the distal end portion of the first implant catheter.

In some examples, the method can comprise advancing the distal end portion of the implant catheter through a main lumen of a shaft of the guide catheter toward a target implantation site, wherein the shaft extends from within and distal to a handle of the guide catheter.

In some examples, the method can comprise, as the distal end portion of the implant catheter is advanced through the main lumen, passively pulling fluid from a fluid reservoir into the main lumen such that a volume of the fluid reservoir decreases.

In some examples, the fluid reservoir is external to the handle and fluidly coupled to the main lumen.

In some examples, the fluid reservoir is internal to a housing of the handle.

In some examples, a method for implanting a prosthetic medical device comprises inserting a shaft of a guide catheter into a vessel of a patient, the shaft having a main lumen and extending from within and distal to a handle of the guide catheter, and inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter. As the distal end portion of the first implant catheter is advanced through the main lumen, fluid is pulled passively from a fluid reservoir into the main lumen such that a volume of the fluid reservoir decreases.

In some examples, a method for implanting a prosthetic medical device comprises inserting a shaft of a guide catheter into a vessel of a patient, the shaft having a main lumen and extending from within and distal to a handle of the guide catheter. The method further comprises inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter. The method further comprises compressing a fluid reservoir disposed within a housing of the handle and flowing fluid from within the fluid reservoir into the main lumen such that a volume of the fluid reservoir decreases.

In some examples, a method for implanting a prosthetic medical device comprises inserting a shaft of a guide catheter into a vessel of a patient, the shaft having a main lumen and extending from within and distal to a handle of the guide catheter. The method further comprises inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter, where, as the distal end portion of the first implant catheter is advanced through the main lumen, fluid is pulled from a fluid reservoir into the main lumen such that a volume of the fluid reservoir decreases, and where the fluid reservoir is disposed within the handle.

In some examples, a method comprises one or more of the features recited in Examples 43-56 and 83-92 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of an exemplary delivery apparatus for a prosthetic heart valve.

FIG. 6 is side view of an exemplary guide catheter configured to receive a delivery apparatus and guide the delivery apparatus through a portion of a patient's vasculature.

FIG. 7A is a cross-sectional side view of the guide catheter of FIG. 6, according to an example.

FIG. 7B is a cross-sectional side view of the guide catheter of FIG. 6, according to an example.

FIG. 8 is a side view of a delivery assembly including the guide catheter of FIG. 6 and the delivery apparatus of FIG. 5.

FIG. 9A is another cross-sectional side view of the guide catheter of FIG. 6 showing a compressible reservoir disposed within a handle of the guide catheter in an expanded configuration as a delivery apparatus is directed through a main lumen of the guide catheter.

FIG. 9B is another cross-sectional side view of the guide catheter of FIG. 9A showing the compressible reservoir in a compressed configuration as the delivery apparatus is directed further along the main lumen of the guide catheter.

FIG. 10A is a cross-sectional side view of an exemplary guide catheter showing an adjustable reservoir external to a handle of the guide catheter in an expanded configuration as a delivery apparatus is directed through a main lumen of the guide catheter.

FIG. 10B is a cross-sectional side view of the guide catheter of FIG. 10A showing the external reservoir in a compressed configuration as the delivery apparatus is directed further along the main lumen of the guide catheter.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Introduction to the Disclosed Technology

As introduced above, a guide catheter can be inserted into a patient's vasculature and then receive an implant delivery apparatus within a main lumen of the guide catheter in order to direct the delivery apparatus therethrough to a target implantation site for a prosthetic implant. In some examples, an inner diameter of the main lumen of the guide catheter and an outer diameter of portions of the implant delivery apparatus can be closely matched. Thus, in some examples, as the delivery apparatus is pushed distally through the main lumen of the guide catheter, a negative pressure (or vacuum) can be created within the main lumen, proximal to the implant, thereby creating an increase in a pressure gradient across one or more fluid seals within a handle of the guide catheter. This can also result in an increase in a force felt by a user as they push the delivery apparatus through the guide catheter (referred to as “push forces”). Accordingly, improvements to the guide catheter that decrease or prevent negative pressure from being created within the main lumen are desirable. Such improvements can, for example, help maintain hemostasis and/or reduce push forces when advancing a delivery apparatus through the guide catheter.

Described herein are various systems, apparatuses, methods, or the like, that, in some examples, can be used in or with delivery apparatuses for prosthetic medical devices (such as prosthetic heart valves or docking devices). In some examples, such systems, apparatuses, and/or methods can provide a fluid reservoir fluidly coupled to a handle of the guide catheter which is configured to provide fluid to a lumen of the guide catheter (and reduce in volume) and maintain a pressure within the lumen as a delivery apparatus is navigated through the lumen of the guide catheter toward an implantation site in a body of a patient. The fluid reservoir can reduce negative pressure created within the system, thereby reducing a pressure gradient across the fluid seals of the handle of the guide catheter and push forces felt by a user pushing the delivery apparatus through the guide catheter. As a result, hemostasis can be maintained, the system can be easier to operate, and, in some instances, can reduce a likelihood of air being pulled into the guide catheter.

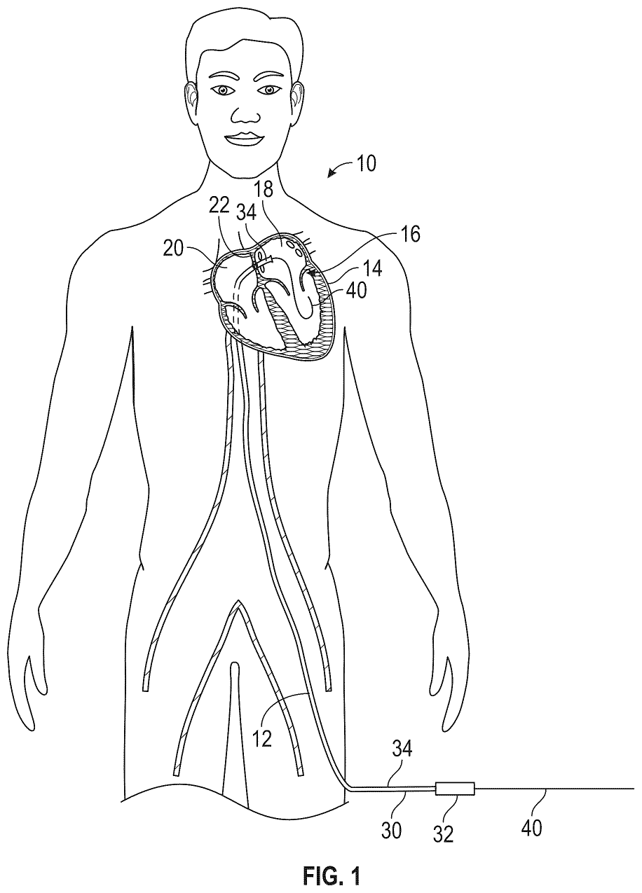

In some examples, the guide catheters disclosed herein can be used to introduce one or more delivery apparatuses (or implant catheters) into the vasculature of a patient and guide the one or more delivery apparatuses at least partially through the vasculature toward a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus. The prosthetic heart valve delivery apparatus is used to deliver a transcatheter prosthetic heart valve inside the docking device.

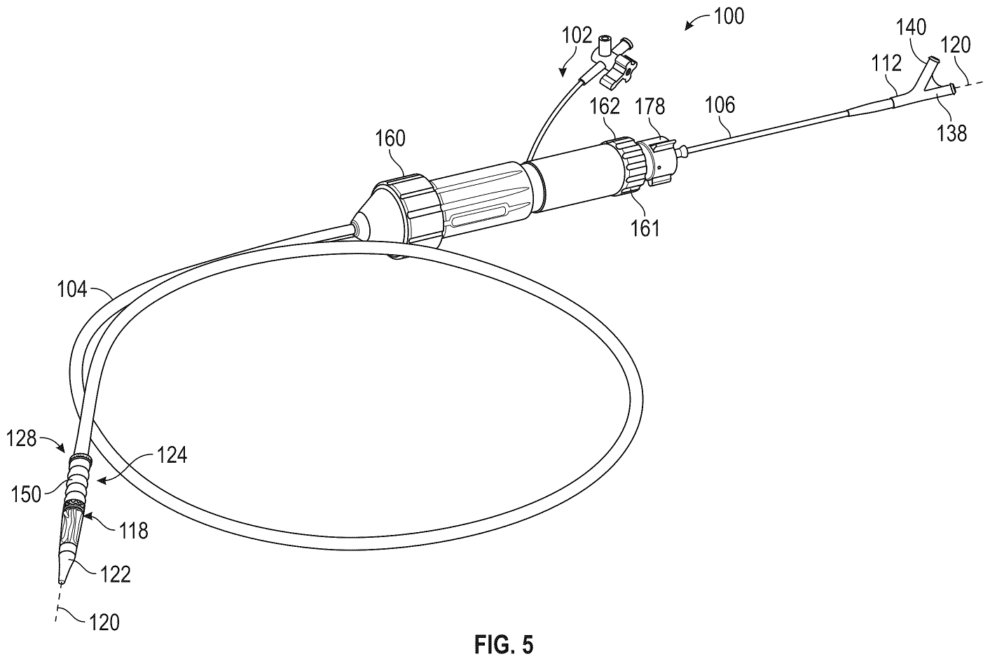

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary delivery apparatus for delivery a prosthetic heart valve within a docking device at a native heart valve is shown in FIG. 5.

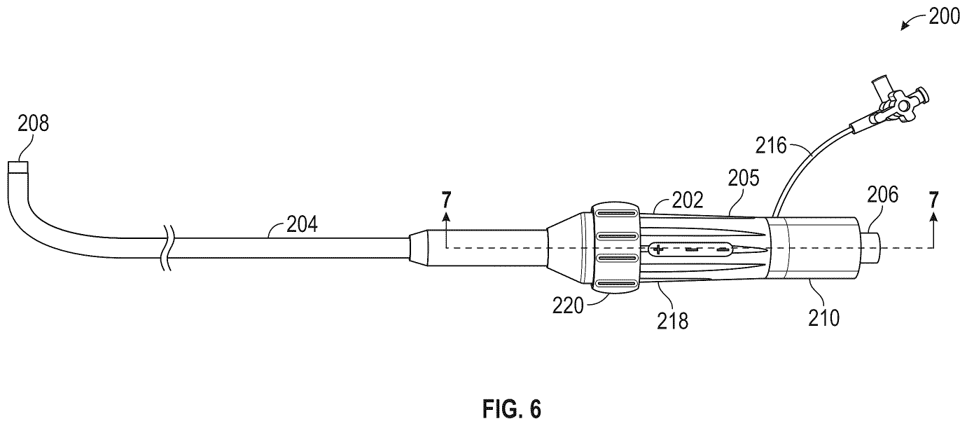

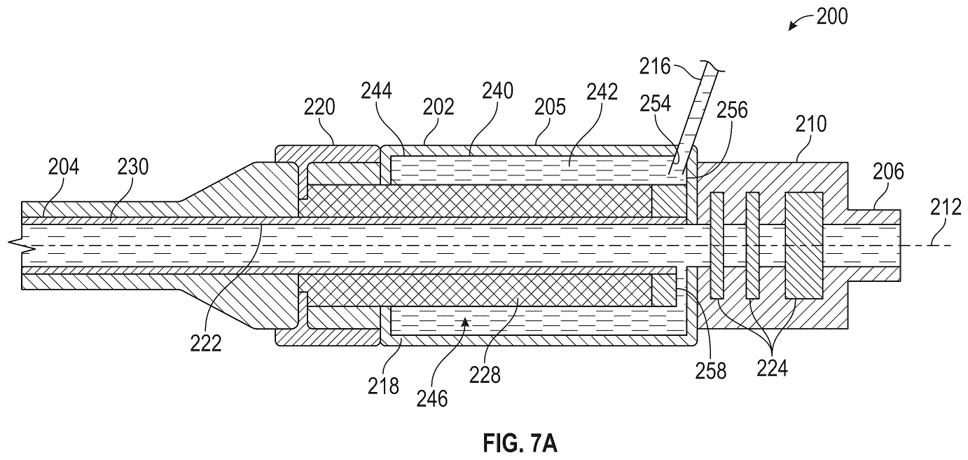

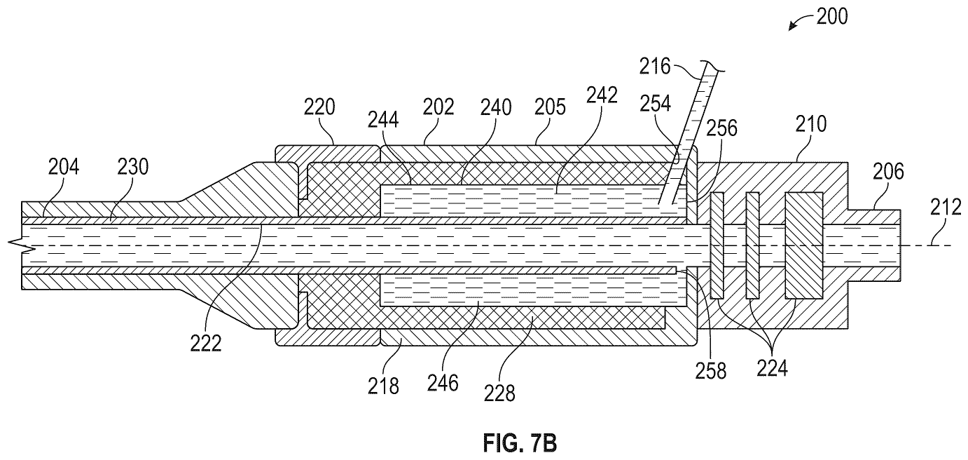

An exemplary guide catheter is shown in more details in FIGS. 6-8. In some examples, as shown in FIGS. 7A, 7B, and 9A-10B, the guide catheter can include a reservoir filled with fluid and fluidly coupled to the main lumen of the guide catheter. As such, the reservoir can compress or reduce in volume and provide fluid to the main lumen as a delivery apparatus travels through the main lumen. As a result, a pressure gradient across one or more seals within a handle of the guide catheter can be reduced, thereby maintaining hemostasis within the guide catheter, reducing a likelihood of air being introduced into the system (in some instances), and reducing push forces felt by a user operating the delivery apparatus.

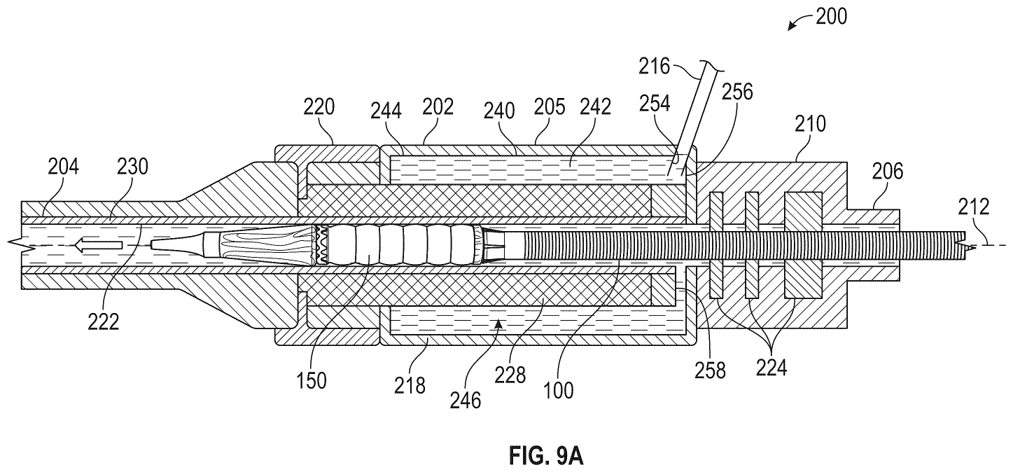

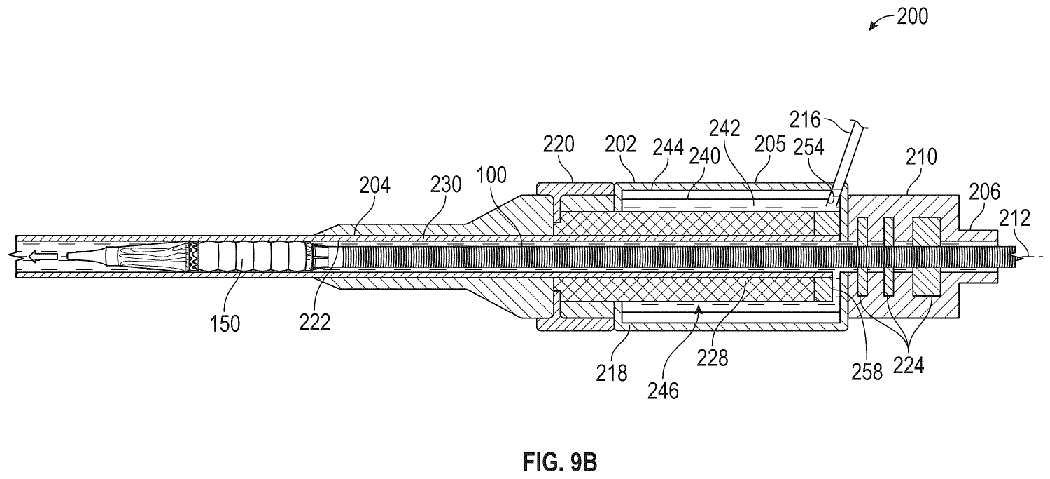

In some examples, the reservoir can be internal to a handle of the guide catheter (FIGS. 7A-7B and 9A-9B).

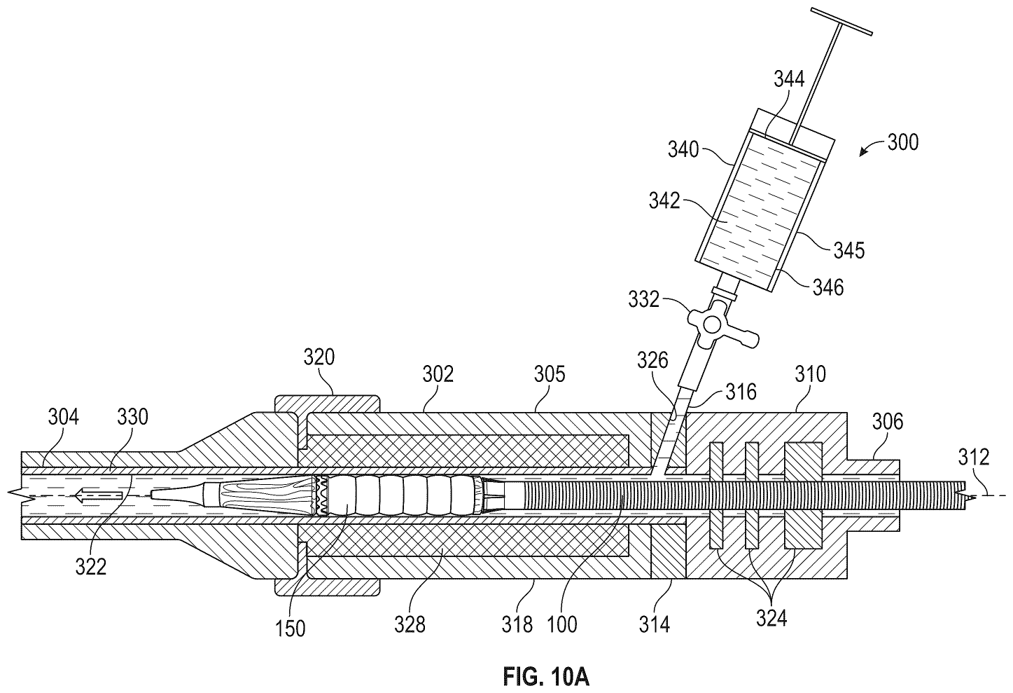

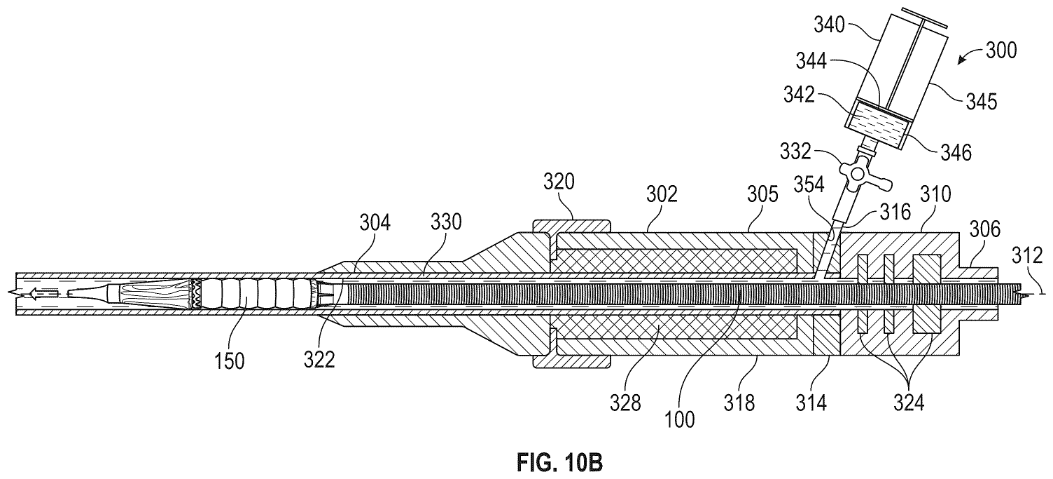

In some examples, the reservoir can be external to a handle of the guide catheter (FIGS. 10A-10B).

### Examples of the Disclosed Technology

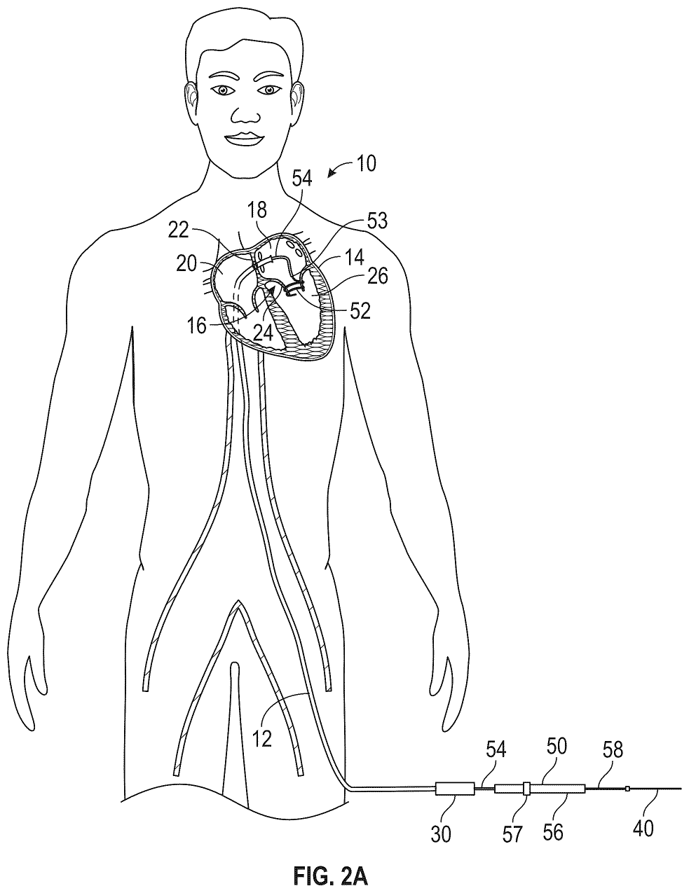

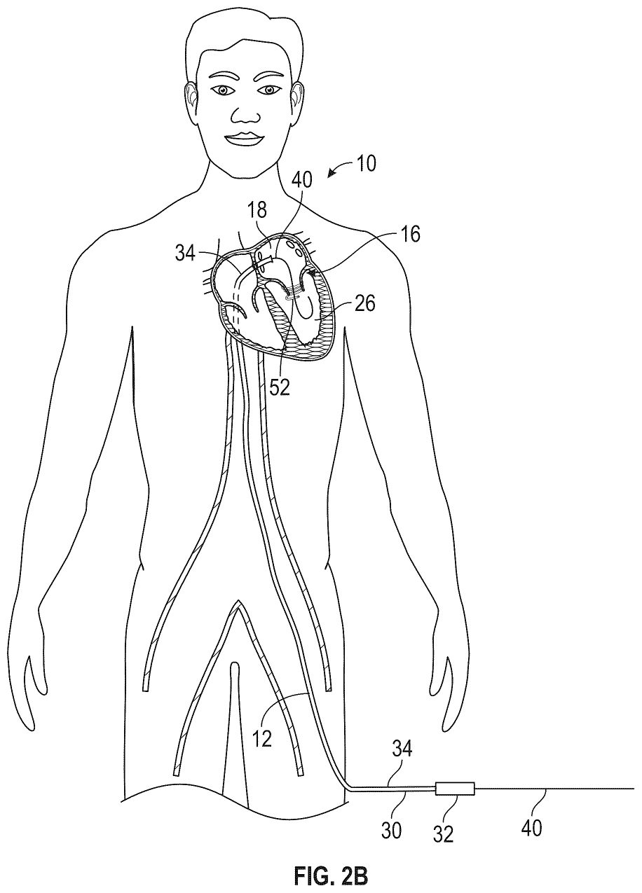

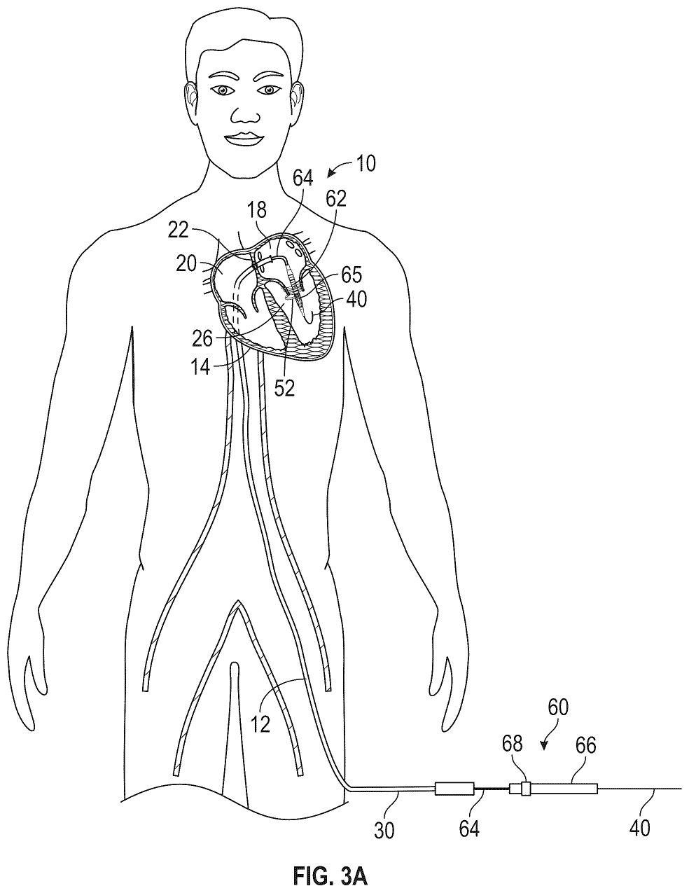

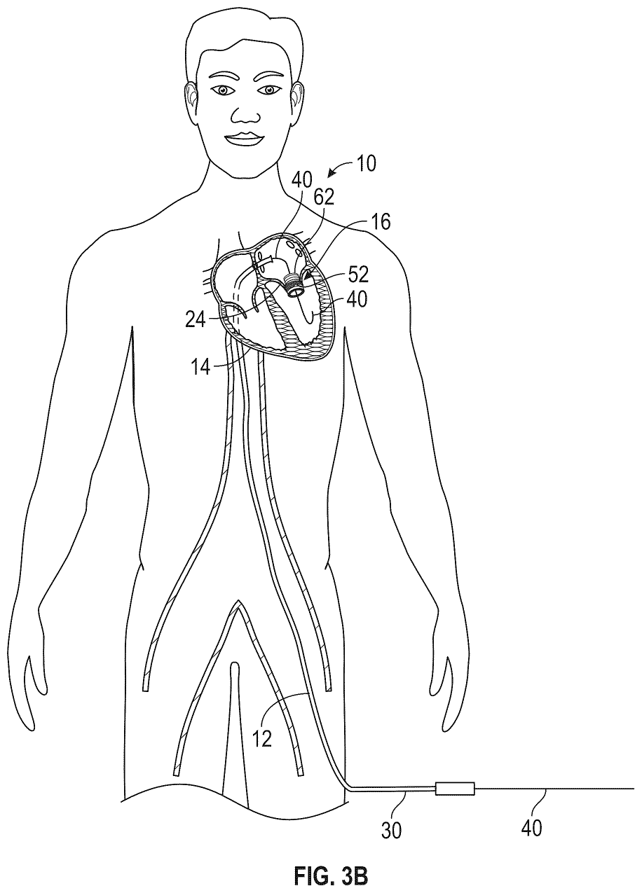

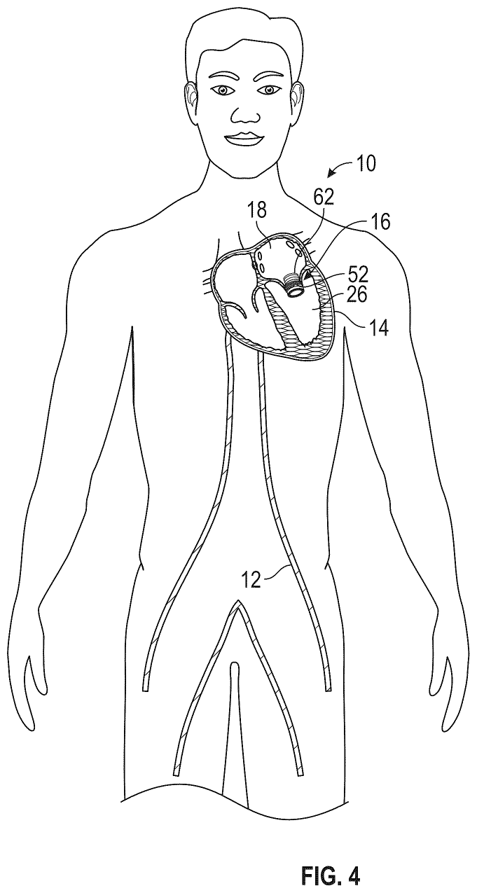

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 20. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can make a small incision in an atrial septum 22 of the heart 14 to allow access to the left atrium 18 from the right atrium 20. The user can insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64.

In some examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and remove the delivery shaft 54 of the docking device delivery apparatus 50 from the patient 10. The user may advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may expand the prosthetic heart valve 62 within the docking device 52 before removing the prosthetic valve delivery apparatus 60 from the patient 10. In some examples, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and through the aortic valve into the left ventricle 26.

FIG. 5 illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve. In some examples, the delivery apparatus 100 is specifically adapted for use in introducing a prosthetic heart valve into a heart.

The delivery apparatus 100 in the illustrated example of FIG. 5 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 (shown in FIG. 5) can overly a distal shoulder of the delivery apparatus 100. As shown in FIG. 5, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 150 (or other medical device) at a fixed position on the balloon 118 during delivery through the patient's vasculature.

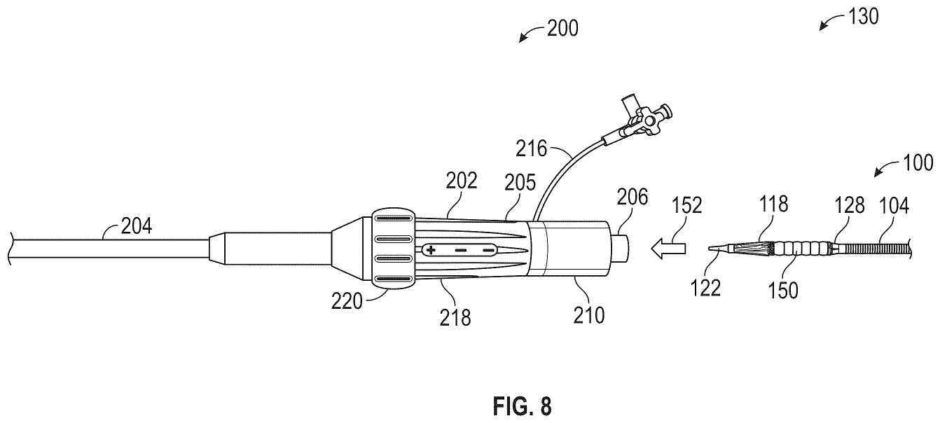

The outer shaft 104 can include a distal tip portion 128 (best seen in FIG. 8) mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 5) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site).

Turning now to FIGS. 6-7B, an exemplary guide catheter, which is referred to below as a guide sheath 200 (and can also be referred to herein as a “delivery apparatus” or an “introducer device” or an “introducer”) is shown. In some examples, the guide sheath 200 can be used in lieu of the guide catheter 30 in a docking device and/or a prosthetic valve implantation procedure, as described above with reference to FIGS. 1-4. The guide sheath 200 can be configured to be inserted into a patient's vasculature and receive an implant catheter or delivery apparatus therein (e.g., such as the delivery apparatus 100 of FIG. 5, as shown in FIG. 8) in order to introduce the implant catheter into the patient's vasculature and at least partially guide the implant catheter therein to a target implantations site. Though the guide sheath 200 is described herein as being used with the delivery apparatus 100, the guide sheath 200 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 200 in the illustrated example comprises a handle 202, an elongated shaft 204 extending distally from the handle 202, and a central longitudinal axis 212 (FIGS. 7A and 7B). The shaft 204 has a main (or primary) lumen 222 that is defined by an inner surface of a wall 230 of the shaft 204 (FIGS. 7A and 7B). The main lumen 222 is configured to receive a delivery apparatus therein (such as any of the prosthetic device delivery apparatuses or implant catheters described herein). In some examples, as shown in FIGS. 7A and 7B, the shaft 204 can extend into the handle 202. Further, in some examples, the main lumen 222 can extend through the handle 202 to an inlet port 206 disposed at a proximal end of the handle 202. Thus, in some examples, an inner surface of a wall of a portion of the handle (e.g., at the proximal end) can further define the main lumen 222. Thus, the main lumen 222 can extend from the inlet port 206 to a distal end 208 of the shaft 204.

The handle 202 can have a housing 205 (also referred to as an “outer housing 205”) comprising a main body portion 218 and a seal housing assembly 210 (which can also be referred to as a “seal stack”) which comprises one or more seals 224 contained therein (FIGS. 7A and 7B). The one or more seals 224 of the seal housing assembly 210 can be configured to fluidly seal the main lumen 222 of the guide sheath 200 from the external environment (e.g., from blood, air, and the like). For example, the one or more seals 224 of the seal housing assembly 210 can be configured to prevent blood from a patient in which the guide sheath 200 is inserted from exiting the guide sheath 200 and prevent air from the environment from entering the guide sheath 200 (e.g., through the inlet port 206). The one or more seals 224 can include a variety of types of seals, such as a duckbill seal, a flapper seal, an umbrella valve, a cross-slit valve, a dome valve, or the like.

The main body portion 218 is disposed adjacent and distal to the seal housing assembly 210. The handle 202 can include a steering mechanism configured to adjust the curvature of the distal end portion of the shaft 204 (as such, the shaft 204 can be referred to as a steerable shaft). In the illustrated example, the handle 202 includes an adjustment member, such as the illustrated rotatable knob 220 (FIGS. 6-7B). The main body portion 218 can house internal flex mechanisms 228 of the guide sheath 200 which are operatively coupled to the rotatable knob 220 (FIGS. 7A and 7B). In some examples, the flex mechanisms 228, and thus the knob 220, can be operatively coupled to the proximal end portion of a pull wire. The pull wire can extend distally from the handle 202 through the shaft 204 and have a distal end portion affixed to the shaft 204 at or near the distal end 208 of the shaft 204. Rotating the knob 220 can increase or decrease the tension in the pull wire, thereby adjusting the curvature of the distal end portion of the shaft 204. Further details on steering or flex mechanisms for a delivery apparatus can be found in U.S. Pat. No. 9,339,384, as already incorporated by reference above.

FIGS. 7A and 7B show two alternate, exemplary locations of the flex mechanisms 228 within the housing 205, including a first location where the flex mechanisms 228 are disposed closer in a radial direction to the shaft 204 (FIG. 7A) and a second location where the flex mechanisms 228 are disposed closer in the radial direction to the housing 205 (and can be spaced away from the shaft 204 by another component) (FIG. 7B), as explained further below.

The handle 202 can include a flush port 216 connected to the housing 205, distal to the seal housing assembly 210. In some examples, the flush port 216 is connected to the main body portion 218 of the housing 205.

The handle 202 further includes a compressible reservoir 240. As shown in FIGS. 7A and 7B, the reservoir 240 is disposed within the housing 205. The reservoir 240 is filled with a fluid 242 and has an adjustable fluid volume. For example, the reservoir 240 can comprise a wall 244 defining an internal cavity 246 which contains the fluid 242 therein. The wall 244 can comprise a flexible material that is configured to expand (or stretch) with increasing fluid volume (of the fluid 242) within the reservoir 240 and compress (or fold or contract) with decreasing fluid volume within the reservoir 240. In some instances, the wall 244 can be configured to conform to the volume of the fluid 242 as the volume of fluid 242 changes, thereby changing a size of the cavity 246. In this way, the reservoir 240 can be configured as an expandable and compressible reservoir, bladder, or balloon with an adjustable (or reducible) fluid volume. As described further below with reference to FIGS. 9A and 9B, the wall 244 of the reservoir 240 can compress as its internal volume of the fluid 242 decreases, thereby decreasing the overall volume of the cavity 246.

The reservoir 240 can be fluidly coupled to the flush port 216 by a first channel 254 (or flush lumen). For example, the first channel 254 can extend from the flush port 216, through the housing 205, and into an interior of the reservoir 240 such that an end of the first channel 254 is disposed within the fluid 242 in the cavity 246. The cavity 246 can be configured to receive fluid from the flush port 216 via the first channel 254 (e.g., during preparation or flushing of the guide sheath 200, as described further below with reference to FIGS. 9A and 9B). Thus, the first channel 254 can serve as and be referred to as a fluid inlet of the reservoir 240.

In some instances, as shown in FIGS. 7A and 7B the first channel 254 can be disposed at a proximal end 256 of the reservoir 240, which is distal and adjacent to the seal housing assembly 210.

In some instances, the first channel 254 can be disposed at another location along the reservoir 240, such as at its distal end or at a location between its proximal end 256 and distal end.

The reservoir 240 can be fluidly coupled to the main lumen 222 via a second channel 258 (or flow lumen). The second channel 258 can extend between, in a radial direction, the cavity 246 of the reservoir 240 and the main lumen 222. During use of the guide sheath 200, the fluid 242 can flow through the second channel 258 into the main lumen 222, based on fluid pressure within the main lumen 222. Thus, the second channel 258 can serve as and be referred to as a fluid outlet of the reservoir 240. In some instances, the second channel 258 can be disposed at the proximal end 256 of the reservoir 240 (FIGS. 7A and 7B).

In some instances, the second channel 258 can be a radially extending channel disposed on one side, or extending circumferentially around only a portion of, the reservoir 240.

In some instances, the second channel 258 can be annular and extend around an entire circumference of the reservoir.

For example, in some instances, the reservoir 240 can be annular and extend circumferentially around the shaft 204 (as shown in FIGS. 7A and 7B). In such instances, the wall 244 of the reservoir 240 can have a radially inward facing portion on one side of the cavity 246 (the side closest to the shaft 204) and a radially outward facing portion on an opposite side of the cavity 246 (the side closest to the housing 205).

In some examples, the reservoir 240 can be disposed radially outside of the flex mechanisms 228 (e.g., radially between the housing 205 and the flex mechanisms 228), as shown in FIG. 7A. In alternative examples, the reservoir 240 can be disposed radially inside the flex mechanisms 228 (e.g., radially between the shaft 204 and the flex mechanisms 228), as shown in FIG. 7B. In both examples, the second channel 258 can be disposed proximal and adjacent to a proximal end of the flex mechanisms 228.

Though the reservoir 240 is shown in FIGS. 7A and 7B and disposed within the housing 205 of the handle 202, in alternative examples, the reservoir 240 can be disposed outside of the housing 205 and fluidly coupled to the main lumen 222 via the second channel 258 extending between the external reservoir 240 and the main lumen 222. The flush port 216 can be attached directly to the reservoir 240.

As introduced above, the guide sheath 200 can be configured to receive a delivery apparatus, such as the delivery apparatus 100 of FIG. 5, within the main lumen 222 of the guide sheath 200. Prior to inserting the delivery apparatus 100 into the guide sheath (and/or prior to inserting the guide sheath into the vasculature of the patient), the main lumen 222 and reservoir 240 can be primed or flushed through the flush port 216. For example, fluid (e.g., fluid 242) can flow through the flush port 216 into the cavity 246 of the reservoir 240 via the first channel 254. In some instances, the cavity 246 can be filled with fluid 242 until the wall 244 of the reservoir 240 expands as far as possible and hits a wall of the housing 205 (FIGS. 7A and 9A) or a wall of the flex mechanisms 228 (FIG. 7B). Once the reservoir 240 is full (and in its expanded configuration, as shown in FIG. 9A), fluid entering the reservoir 240 from the flush port 215 can continue to flow into the main lumen 222 via the second channel 258. In some instances, this process can continue until the main lumen 222 is filled to a desired level. In some examples, the fluid 242 used to fill the reservoir 240 and main lumen 222 is saline or an alternate biocompatible flush fluid.

After positioning the shaft 204 of the guide sheath 200 within the vasculature of a patient, a distal end portion of the delivery apparatus 100 (e.g., the nose cone 122 and radially compressed prosthetic heart valve 150) can be inserted into the inlet port 206 of the handle 202 of the guide sheath 200, as indicated by arrow 152 in FIG. 8. The distal end portion of the delivery apparatus 100 can be navigated through the seal housing assembly 210 and into the main lumen 222 of the guide sheath 200. The delivery apparatus 100 can continue to be navigated through the main lumen 222 of the shaft 204, toward the implantation site. The assembly shown in FIG. 8 can be referred to as a delivery assembly 130.

FIGS. 9A and 9B depict different exemplary states of the reservoir 240 as the delivery apparatus 100 is navigated through the main lumen 222 of the guide sheath 200. As introduced above, in FIG. 9A, the reservoir 240 is in an expanded state or configuration. The distal end portion of the delivery apparatus 100, including the prosthetic heart valve 150, has just been inserted into the main lumen 222 and is disposed within the handle 202.

As the distal end portion of the delivery apparatus 100 is navigated further through the main lumen 222, pressure (fluid pressure) in a proximal portion of the main lumen 222 (e.g., the portion within the handle 202 and shown in FIG. 9A) can decrease due to the prosthetic heart valve 150 (or alternate portions of the delivery apparatus 100) sliding against the inner surface of the wall 230 of the shaft 204. As the pressure within the main lumen 222 decreases, the fluid 242 within the reservoir 240 can be pulled from the cavity 246 of the reservoir into the main lumen 222, and at the same time the wall 244 of the reservoir 240 compresses radially inward, thereby decreasing the internal volume of the cavity 246 of the reservoir 240 (as shown in FIG. 9A). As a result, the pressure within the main lumen 222 can equalize, and in some instances, a negative pressure (or vacuum) created by advancing the distal end portion of the delivery apparatus 100 through the main lumen 222 can be reduced.

It should be noted that during this process, no fluid is entering the reservoir via the first channel 254 (e.g., the fluid inlet to the reservoir 240 may be closed).

In the expanded state of the reservoir 240 (FIG. 9A), the cavity 246 has a first fluid volume. In a compressed state or configuration of the reservoir 240 (FIG. 9B), the cavity 246 has a second fluid volume that is smaller than the first fluid volume. Further, in the compressed state, the radially outward facing portion of the wall 244 of the reservoir 240 is spaced away from an inner surface of a wall of the housing 205. In this way, the overall volume of the cavity 246 decreases along with the decreasing fluid volume, as fluid flows out of the cavity 246 and into the main lumen 222 of the guide sheath 200.

In some instances, a size (e.g., diameter, width, and/or length) of the second channel 258 can be specified based on a selected fluid volume transfer rate that maintains pressure within the main lumen 222 at a desired level (e.g., a non-negative pressure) and maintains hemostasis within the guide sheath. For example, by increasing a volume of the second channel 258, the fluid transfer rate between the reservoir 240 and the main lumen 222 can increase, thereby better maintaining the pressure within the main lumen 222 at a non-negative value as the delivery apparatus is navigated through the main lumen 222.

In some examples, by maintaining the pressure within the main lumen of the guide sheath or catheter at a non-negative level by using the compressible reservoir, push forces felt by a user navigating the delivery apparatus through the main lumen of the guide catheter can also be reduced.

FIGS. 10A and 10B depict an exemplary guide catheter or guide sheath 300 (which can also be referred to herein as a “delivery apparatus” or an “introducer device” or an “introducer”). In some examples, the guide sheath 300 can be used in lieu of the guide catheter 30 in a docking device and/or a prosthetic valve implantation procedure, as described above with reference to FIGS. 1-4. The guide sheath 300 can be configured to be inserted into a patient's vasculature and receive an implant catheter or delivery apparatus therein (e.g., such as the delivery apparatus 100 of FIG. 5) in order to introduce the implant catheter into the patient's vasculature and at least partially guide the implant catheter therein to a target implantation site. For example, in some instances, the guide sheath 300 can be used in lieu of the guide sheath 200 in FIG. 8.

Although the guide sheath 300 is described herein as being used with the delivery apparatus 100, the guide sheath 300 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 300 can be similar to the guide sheath 200 except it comprises a reservoir 340 that is disposed external to a handle 302 of the guide sheath (and thus can also be referred to herein as an “external reservoir 340”).

For example, the guide sheath 300 can comprise a handle 302, an elongated shaft 304 extending distally from the handle 302, and a central longitudinal axis 312. The shaft 304 has a main (or primary) lumen 322 that is defined by an inner surface of a wall 330 of the shaft 304. The main lumen 322 is configured to receive a delivery apparatus therein (such as any of the prosthetic device delivery apparatuses or implant catheters described herein). In some examples, as shown in FIGS. 10A and 10B, the shaft 304 can extend into the handle 302. Further, in some examples, the main lumen 322 can extend through the handle 302 to an inlet port 306 disposed at a proximal end of the handle 302. Thus, in some examples, an inner surface of a wall of a portion of the handle (e.g., at the proximal end) can further define the main lumen 322. Thus, the main lumen 322 can extend from the inlet port 306 to a distal end (e.g., distal end 208 shown in FIG. 6) of the shaft 304.

The handle 302 can have a housing 305 (also referred to as an “outer housing 305”) comprising a main body portion 318 and a seal housing assembly 310 (which can also be referred to as a “seal stack”) which comprises one or more seals 324 contained therein. The one or more seals 324 of the seal housing assembly 310 can be configured to fluidly seal the main lumen 322 of the guide sheath 300 from the external environment. For example, the one or more seals 324 of the seal housing assembly 310 can be configured to prevent blood from a patient in which the guide sheath 300 is inserted from exiting the guide sheath 300 and prevent air from the environment from entering the guide sheath 300 (e.g., through the inlet port 306). The one or more seals 324 can include a variety of types of seals, such as a duckbill seal, a flapper seal, an umbrella valve, a cross-slit valve, a dome valve, or the like.

A flush port 316 can be connected to the outer housing 305.

The handle 302 can, in some instances, include an adaptor spine 314 disposed adjacent and distal to the seal housing assembly 310. In some examples, the flush port 316 is connected to the outer housing 305 at the adaptor spine 314. A flush lumen 326 (or fluid channel) of the adaptor spine 314 is connected to the flush port 316 and further connects to the main lumen 322. The flush port 316 can be configured to receive fluid through a lumen thereof. In this way, the flush port 316 can be fluidly coupled to the main lumen 322 by the flush lumen 326.

The handle 302 can include a steering mechanism configured to adjust the curvature of the distal end portion of the shaft 304 (as such, the shaft 304 can be referred to as a steerable shaft). In the illustrated example, the handle 302 includes an adjustment member, such as the illustrated rotatable knob 320. The main body portion 318 can house internal flex mechanisms 328 of the guide sheath 300 which are operatively coupled to the rotatable knob 320. In some examples, the flex mechanisms 328, and thus the knob 320, can be operatively coupled to the proximal end portion of a pull wire. The pull wire can extend distally from the handle 302 through the shaft 304 and have a distal end portion affixed to the shaft 304 at or near the distal end of the shaft 304. Rotating the knob 320 can increase or decrease the tension in the pull wire, thereby adjusting the curvature of the distal end portion of the shaft 304. Further details on steering or flex mechanisms for a delivery apparatus can be found in U.S. Pat. No. 9,339,384, as already incorporated by reference above.

The guide sheath 300 further includes a reservoir 340 with an adjustable volume. As shown in FIGS. 10A and 10B, the reservoir 340 is disposed external to the housing 305 of the handle 302. The reservoir 340 can be fluidly coupled to the flush port 316, and thus the flush lumen 326 and the main lumen 322.

For example, in some instances, the reservoir 340 can be directly coupled to the flush port 316.

In some instances, the reservoir 340 can be coupled to the flush port 316 at a stopcock 332 or another type of connector or adaptor that can open and close the fluid connection between the reservoir 340 and the flush port 316. For example, when the stopcock 332 is in an open position, as shown in FIGS. 10A and 10B, fluid can be transferred (and flow) from the reservoir 340 to the flush port 316, flush lumen 326, and into the main lumen 322.

The reservoir 340 is filled with a fluid 342 and has an adjustable fluid volume. For example, the reservoir 340 can comprise a barrel 345 and a movable wall 344 (also referred to as a plunger) disposed within the barrel 345. The barrel 345 and the movable wall 344 define an internal cavity 346 which contains the fluid 342 therein.

In some examples, the reservoir 340 is a syringe.

In some instances, as the movable wall 344 is pushed into the barrel 345, fluid exits the reservoir 340 and the fluid volume of the fluid 342 decreases. In some instances, if the fluid 342 is pulled into the flush port 316 (e.g., from a negative pressure inside the main lumen 322, as explained further below), the movable wall 344 can be pulled with the fluid 342 and the fluid volume of the fluid 342 within the reservoir 340 (and the volume of the cavity 346) decreases.

In this way, the reservoir 340 can be configured as an expandable and contractable (or reducible) reservoir (via movement of the movable wall 344) with an adjustable (or reducible) fluid volume.

During use of the guide sheath 300, the reservoir 340 can be open and fluidly connected to the main lumen 322 via the flush lumen 326 (e.g., by opening the stopcock 332). As a result, the fluid 342 inside the reservoir 340 can freely and passively flow through flush lumen 326 into the main lumen 322 based on fluid pressure within the main lumen 322. As used herein, “passively” or “passive” in reference to the flow of fluid from the various reservoirs into the main lumen of the guide sheath can refer to the flow of fluid without user intervention. For example, fluid is pulled (passively) into the main lumen from the reservoir due to a vacuum or negative pressure in the main lumen, and not due to a user actively pushing fluid from the reservoir.

As introduced above, the guide sheath 300 can be configured to receive a delivery apparatus, such as the delivery apparatus 100 of FIG. 5, within the main lumen 322 of the guide sheath 300. Prior to inserting the delivery apparatus 100 into the guide sheath 300 (and/or prior to inserting the guide sheath into the vasculature of the patient), the reservoir 340 can be filled with fluid 342.

In some instances, the flush lumen 326 and main lumen 322 can be primed or flushed through the flush port 316 with the reservoir 340 or another flushing device. For example, fluid (e.g., fluid 342) can flow through the flush port 316 and flush lumen 326 into the main lumen 322. The reservoir 340 can be relatively full and in its expanded configuration, as shown in FIG. 10A.

After positioning the shaft 304 of the guide sheath 300 within the vasculature of a patient, a distal end portion of the delivery apparatus 100 (e.g., the nose cone 122 and radially compressed prosthetic heart valve 150) can be inserted into the inlet port 306 of the handle 302 of the guide sheath 300. The distal end portion of the delivery apparatus 100 can be navigated through the seal housing assembly 310 and into the main lumen 322 of the guide sheath 300.

In some examples, the reservoir 340 can be attached to the flush port 316 after the delivery apparatus 100 is inserted into the guide sheath 300 (e.g., after the distal end of the delivery apparatus 100 is inserted past, or distal to, the seal housing assembly 310). The reservoir 340 can then be opened to the flush port 316 such that fluid can passively be pulled into the flush lumen 326 and main lumen 322 from the reservoir 340.

The delivery apparatus 100 can continue to be navigated through the main lumen 322 of the shaft 304, toward the implantation site.

FIGS. 10A and 10B depict different exemplary states of the reservoir 340 as the delivery apparatus 100 is navigated through the main lumen 322 of the guide sheath 300. As introduced above, in FIG. 10A, the reservoir 340 is in an expanded state or configuration. The distal end portion of the delivery apparatus 100, including the prosthetic heart valve 150, has just been inserted into the main lumen 322 and is disposed within the handle 302.

As the distal end portion of the delivery apparatus 100 is navigated further through the main lumen 322, pressure (fluid pressure) in a proximal portion of the main lumen 322 (e.g., the portion within the handle 302 and shown in FIG. 10A) can decrease due to the prosthetic heart valve 150 (or alternate portions of the delivery apparatus 100) sliding against the inner surface of the wall 330 of the shaft 304 (e.g., since the valve 150 sliding against the inner surface of the wall 330 prevents fluid from flowing distal to the valve 150). As the pressure within the proximal portion of the main lumen 322 decreases, the fluid 342 within the reservoir 340 can be pulled from the cavity 346 of the reservoir 340 into the main lumen 322. The wall 344 of the reservoir 340 can move inward toward the fluid 342 and flush port 316, thereby decreasing the internal volume of the cavity 346 of the reservoir 340 (as shown in FIG. 10B). As a result, the pressure within the main lumen 322 can equalize, and in some instances, a negative pressure (or vacuum) created by advancing the distal end portion of the delivery apparatus 100 through the main lumen 322 can be reduced.

In the expanded state of the reservoir 340 (FIG. 10A), the cavity 346 has a first fluid volume. In a compressed state or configuration of the reservoir 340 (FIG. 10B), the cavity 346 has a second fluid volume that is smaller than the first fluid volume. Further, in the compressed or reduced state, the movable wall 344 of the reservoir 340 is pushed inward toward an outlet of the reservoir 340 (where the reservoir 340 couples to the flush port 316). In this way, the overall volume of the cavity 346 can decrease along with the decreasing fluid volume, as fluid flows out of the cavity 346 and into the main lumen 322 of the guide sheath 300.

It should be noted that fluid within the main lumen 322 (and the main lumens shown in the other figures, such as FIGS. 9A and 9B) and the various reservoirs is depicted with a generic cross-hatching. However, fluid downstream or distal to the prosthetic heart valve of the delivery apparatus inside the guide sheath can be a combination of priming fluid (e.g., saline) and blood, while fluid upstream of the prosthetic heart valve of the delivery apparatus can be fluid provided from the reservoir (e.g., saline).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle comprising: a housing; and a compressible reservoir disposed within the housing, wherein the reservoir is filled with fluid and has an adjustable fluid volume; and a shaft extending within and distally from the handle and having a main lumen, wherein the main lumen is fluidly coupled to the reservoir by a first channel disposed within the handle.

Example 2. The delivery apparatus of any example herein, particularly example 1, wherein a wall of the reservoir comprises a flexible material, and wherein the reservoir is configured to compress as its fluid volume reduces.

Example 3. The delivery apparatus of any example herein, particularly either example 1 or example 2, wherein the handle further comprises a flush port coupled to the housing, and wherein the flush port is fluidly coupled to the reservoir by a second channel.

Example 4. The delivery apparatus of any example herein, particularly example 3, wherein the handle further comprises a seal housing assembly including one or more fluid seals, and wherein the first channel is disposed at a proximal end of the reservoir that is disposed adjacent to the seal housing assembly.

Example 5. The delivery apparatus of any example herein, particularly example 4, wherein the flush port and the second channel are disposed at the proximal end of the reservoir that is disposed adjacent to the seal housing assembly.

Example 6. The delivery apparatus of any example herein, particularly any one of examples 3-5, wherein the second channel extends from the flush port and radially inward into an interior cavity of the reservoir.

Example 7. The delivery apparatus of any example herein, particularly any one of examples 1-6, wherein the reservoir is annular and extends axially along a portion of the handle, and wherein the reservoir is disposed radially outward of the shaft, relative to a central longitudinal axis of the delivery apparatus.

Example 8. The delivery apparatus of any example herein, particularly any one of examples 1-7, wherein the reservoir is disposed between, in a radial direction, the housing and flex mechanisms of the handle, wherein the flex mechanisms are configured to adjust a curvature of a distal end portion of the shaft.

Example 9. The delivery apparatus of any example herein, particularly any one of examples 1-7, wherein the reservoir is disposed between, in a radial direction, the shaft and flex mechanisms of the handle, wherein the flex mechanisms are disposed within the housing and configured to adjust a curvature of a distal end portion of the shaft.

Example 10. The delivery apparatus of any example herein, particularly either example 8 or example 9, wherein the handle further comprises a rotatable knob operatively coupled to the flex mechanisms.

Example 11. The delivery apparatus of any example herein, particularly any one of examples 1-10, wherein the fluid inside the reservoir is saline.

Example 12. The delivery apparatus of any example herein, particularly any one of examples 1-11, wherein the reservoir is configured to compress radially inward toward the main lumen and decrease in volume as fluid within the reservoir is transferred to the main lumen via the first channel.

Example 13. A delivery assembly comprising: an implant catheter; and a guide catheter comprising: a handle comprising: a housing and a flush port coupled to the housing; and a compressible reservoir disposed within the housing, wherein the reservoir is filled with fluid and has an adjustable fluid volume, and wherein the flush port is fluidly coupled to the reservoir; and a shaft extending distally from within the handle and having a main lumen that is configured to receive a portion of the implant catheter therethrough, wherein the main lumen is fluidly coupled to the reservoir.

Example 14. The delivery assembly of any example herein, particularly example 13, wherein the reservoir is disposed around and radially outward of the shaft, and wherein the main lumen is fluidly coupled to the reservoir by a first channel extending between the main lumen and the reservoir.

Example 15. The delivery assembly of any example herein, particularly either example 13 or example 14, wherein the reservoir is annular and disposed between the main lumen and the housing.

Example 16. The delivery assembly of any example herein, particularly either example 14 or example 15, wherein the handle further comprises a seal housing assembly including one or more fluid seals, and wherein the sealing housing assembly is disposed at a proximal end of handle.

Example 17. The delivery assembly of any example herein, particularly example 16, wherein the reservoir is disposed adjacent and distal to the seal housing assembly within the handle, and wherein the first channel is disposed at a proximal end of the reservoir, adjacent to the seal housing assembly.

Example 18. The delivery assembly of any example herein, particularly either example 16 or example 17, wherein the flush port is fluidly coupled to the reservoir by a second channel extending from the flush port and into an interior cavity of the reservoir that is filled with the fluid.

Example 19. The delivery assembly of any example herein, particularly example 18, wherein the second channel is disposed at a proximal end of the reservoir, adjacent to the seal housing assembly.

Example 20. The delivery assembly of any example herein, particularly any one of examples 16-19, wherein the handle further comprises a flex mechanism disposed within the housing and surrounding a portion of the shaft, and wherein the flex mechanism is configured to adjust a curvature of a distal end portion of the shaft.

Example 21. The delivery assembly of any example herein, particularly example 20, wherein the handle further comprises a rotatable knob operatively coupled to the flex mechanism, and wherein the reservoir is disposed between, in an axial direction, the knob and the seal housing assembly.

Example 22. The delivery assembly of any example herein, particularly example 21, wherein the reservoir is further disposed around the flex mechanism.

Example 23. The delivery assembly of any example herein, particularly any one of examples 13-22, wherein the reservoir is configured to compress radially inward toward the main lumen and decrease in volume as fluid within the reservoir is transferred to the main lumen.

Example 24. A guide sheath comprising: a handle comprising: a housing and a flush port coupled to the housing; a seal housing assembly including one or more fluid seals; and a compressible reservoir disposed within the housing, distal to the seal housing assembly, wherein the reservoir is filled with fluid and has an adjustable volume, and wherein the flush port is fluidly coupled to the reservoir; and a shaft extending within and distally from the handle and having a main lumen that extends within the housing and through the seal housing assembly, wherein the main lumen is fluidly coupled to the reservoir.

Example 25. The guide sheath of any example herein, particularly example 24, wherein the reservoir comprises a flexible wall and a cavity defined by the wall, wherein the cavity is filled with the fluid, and wherein the wall is configured to move away from the housing and toward the shaft as fluid from the cavity flows into the main lumen.

Example 26. The guide sheath of any example herein, particularly example 25, wherein the reservoir comprises a fluid inlet configured as a first channel that extends between the flush port and the cavity, and wherein the reservoir comprises a fluid outlet configured as a second channel that extends between the cavity and the main lumen.

Example 27. The guide sheath of any example herein, particularly example 26, wherein the first channel is disposed at a proximal end of the reservoir that is disposed adjacent to the seal housing assembly.

Example 28. The guide sheath of any example herein, particularly either example 26 or example 27, wherein the second channel is disposed at a proximal end of the reservoir that is disposed adjacent to the seal housing assembly.

Example 29. The guide sheath of any example herein, particularly any one of examples 24-28, wherein the handle further comprises a flex mechanism that is configured to adjust a curvature of a distal end portion of the shaft and disposed within the housing, and wherein the flex mechanism is disposed distal to the seal housing assembly.

Example 30. The guide sheath of any example herein, particularly example 29, wherein the reservoir is disposed radially outside of the flex mechanism.

Example 31. The guide sheath of any example herein, particularly example 29, wherein the reservoir is disposed radially inside of the flex mechanism.

Example 32. The guide sheath of any example herein, particularly any one of examples 29-31, wherein the handle further comprises a rotatable knob operatively coupled to the flex mechanism, and wherein the rotatable knob is disposed distal to the reservoir.

Example 33. The guide sheath of any example herein, particularly any one of examples 24-32, wherein the reservoir is annular and extends axially along a portion of the handle, and wherein the reservoir is disposed radially outward of the shaft, relative to a central longitudinal axis of the guide sheath.

Example 34. A delivery apparatus comprising: a handle; a shaft extending within and distally from the handle and having a main lumen; and a bladder containing a fluid volume that is fluidly coupled to the main lumen, wherein the bladder comprises a flexible wall that is configured to conform to the fluid volume and contract inward as the fluid volume decreases.

Example 35. The delivery apparatus of any example herein, particularly example 34, wherein the bladder is annular, and wherein a radially outward facing portion of the wall which extends around a circumference of the shaft is configured to contract inward as the fluid volume decreases.

Example 36. The delivery apparatus of any example herein, particularly either example 34 or example 35, wherein the bladder has an expanded state and a compressed state, and wherein the fluid volume is larger in the expanded state than in the compressed state.

Example 37. The delivery apparatus of any example herein, particularly any one of examples 34-36, wherein the bladder is disposed internal to a housing of the handle.

Example 38. The delivery apparatus of any example herein, particularly example 37, wherein the bladder has an expanded state and a compressed state, wherein in the expanded state the wall of the bladder is disposed closer to the housing than in the compressed state, and wherein in the compressed state the wall of the bladder is disposed away from the housing and toward the shaft.

Example 39. The delivery apparatus of any example herein, particularly either example 37 or example 38, wherein the bladder is annular and disposed around the shaft.

Example 40. The delivery apparatus of any example herein, particularly any one of examples 34-36, wherein the bladder is disposed external to a housing of the handle.

Example 41. The delivery apparatus of any example herein, particularly any one of examples 34-40, wherein the bladder includes a fluid outlet that is fluidly coupled to the main lumen, and wherein the bladder includes a fluid inlet.

Example 42. The delivery apparatus of any example herein, particularly any one of examples 34-41, wherein the handle comprises one or more fluid seals disposed at a proximal end of the handle, wherein the main lumen extends through the one or more fluid seals, and wherein the bladder is fluidly coupled to the main lumen at a location that is distal to the one or more fluid seals.

Example 43. A method for implanting a prosthetic medical device, comprising: inserting a shaft of a guide catheter into a vessel of a patient, the shaft having a main lumen and extending from within and distal to a handle of the guide catheter; inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter; and compressing a fluid reservoir disposed within a housing of the handle and flowing fluid from within the fluid reservoir into the main lumen such that a volume of the fluid reservoir decreases.

Example 44. The method of any example herein, particularly example 43, wherein the compressing the fluid reservoir occurs passively as fluid from within the fluid reservoir is pulled into the main lumen by decreasing fluid pressure within the main lumen as the distal end portion of the first implant catheter is advanced further along the main lumen.

Example 45. The method of any example herein, particularly either example 43 or example 44, wherein the compressing the fluid reservoir includes contracting a wall of the fluid reservoir inward such that an internal cavity of the fluid reservoir that contains the fluid decreases as the fluid flows into the main lumen.

Example 46. The method of any example herein, particularly any one of examples 43-45, wherein flowing fluid from within the reservoir into the main lumen includes flowing fluid from within the reservoir through a fluid channel extending between an interior of the fluid reservoir and the main lumen inside the housing.

Example 47. The method of any example herein, particularly any one of examples 43-46, wherein the handle includes a flush port coupled to the housing and disposed distal to one or more fluid seals of the handle that are disposed adjacent to a proximal end of the guide catheter, and wherein the fluid reservoir is fluidly coupled to the flush port.

Example 48. The method of any example herein, particularly any one of examples 43-47, further comprising implanting the prosthetic medical device at the target implantation site, removing the first implant catheter from the guide catheter, and inserting a second implant catheter into the guide catheter and advancing the second implant catheter through the main lumen toward the target implantation site.

Example 49. The method of any example herein, particularly example 48, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 50. A method for implanting a prosthetic medical device, comprising: inserting a shaft of a guide catheter into a vessel of a patient, the shaft having a main lumen and extending from within and distal to a handle of the guide catheter; and inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter, wherein, as the distal end portion of the first implant catheter is advanced through the main lumen, fluid is pulled from a fluid reservoir into the main lumen such that a volume of the fluid reservoir decreases, and wherein the fluid reservoir is disposed within the handle.

Example 51. The method of any example herein, particularly example 50, wherein the fluid reservoir is disposed around the shaft.

Example 52. The method of any example herein, particularly example 51, wherein as fluid is pulled from the fluid reservoir into the main lumen, a wall of the fluid reservoir contracts inward toward the main lumen of the shaft and a fluid cavity containing the fluid and defined by the wall of the fluid reservoir decreases in size.

Example 53. The method of any example herein, particularly either example 51 or example 52, wherein the fluid reservoir is fluidly coupled to the main lumen by a first channel disposed within the handle.

Example 54. The method of any example herein, particularly any one of examples 50-53, wherein the handle includes a flush port coupled to a housing of the handle and disposed distal to one or more fluid seals of the handle that are disposed adjacent to a proximal end of the guide catheter, and wherein the fluid reservoir is fluidly coupled to the flush port.

Example 55. The method of any example herein, particularly any one of examples 50-54, further comprising implanting the prosthetic medical device at the target implantation site, removing the first implant catheter from the guide catheter, and inserting a second implant catheter into the guide catheter and advancing the second implant catheter through the main lumen toward the target implantation site.

Example 56. The method of any example herein, particularly example 55, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 57. A delivery apparatus comprising: a handle; a shaft extending within and distally from the handle and having a main lumen; and a reservoir fluidly coupled to the main lumen, wherein the reservoir is filled with fluid and has an adjustable fluid volume, and wherein the reservoir is configured to passively supply fluid to the main lumen based on a fluid pressure in the main lumen.

Example 58. The delivery apparatus of any example herein, particularly example 57, wherein the handle further comprises a flush port coupled to a housing of the handle, and wherein the flush port is fluidly coupled to the reservoir.

Example 59. The delivery apparatus of any example herein, particularly example 58, wherein the handle further comprises a seal housing assembly including one or more fluid seals, and wherein the flush port is disposed distal and adjacent to the seal housing assembly.

Example 60. The delivery apparatus of any example herein, particularly any one of examples 57-59, wherein the main lumen is fluidly coupled to the reservoir by a first channel disposed within the handle.

Example 61. The delivery apparatus of any example herein, particularly example 60, wherein the reservoir comprises a movable wall that is configured to conform to the adjustable fluid volume and move inward as fluid within the reservoir is transferred to the main lumen via the first channel and the fluid volume decreases.

Example 62. The delivery apparatus of any example herein, particularly any one of examples 57-61, wherein the handle comprises a housing and the reservoir is disposed internal to the housing.

Example 63. The delivery apparatus of any example herein, particularly example 62, wherein a wall of the reservoir comprises a flexible material, and wherein the reservoir is configured to compress as its fluid volume reduces.

Example 64. The delivery apparatus of any example herein, particularly either example 62 or 63, wherein the handle further comprises a flush port coupled to a housing of the handle, and wherein a second channel extends from the flush port and radially inward into an interior cavity of the reservoir.

Example 65. The delivery apparatus of any example herein, particularly any one of examples 62-64, wherein the reservoir is annular and extends axially along a portion of the handle, and wherein the reservoir is disposed radially outward of the shaft, relative to a central longitudinal axis of the delivery apparatus.

Example 66. The delivery apparatus of any example herein, particularly any one of examples 62-65, wherein the reservoir is disposed between, in a radial direction, the housing and flex mechanisms of the handle, wherein the flex mechanisms are configured to adjust a curvature of a distal end portion of the shaft.

Example 67. The delivery apparatus of any example herein, particularly any one of example 62-65, wherein the reservoir is disposed between, in a radial direction, the shaft and flex mechanisms of the handle, wherein the flex mechanisms are disposed within the housing and configured to adjust a curvature of a distal end portion of the shaft.

Example 68. The delivery apparatus of any example herein, particularly either example 66 or 67, wherein the handle further comprises a rotatable knob operatively coupled to the flex mechanisms.

Example 69. The delivery apparatus of any example herein, particularly any one of examples 62-68, wherein the reservoir is configured to compress radially inward toward the main lumen and decrease in volume as fluid within the reservoir is transferred to the main lumen via a first channel.

Example 70. The delivery apparatus of any example herein, particularly any one of examples 57-61, wherein the handle comprises a housing and the reservoir is disposed external to the housing.

Example 71. The delivery apparatus of any example herein, particularly example 70, wherein the reservoir is fluidly coupled to a flush port that is coupled to the housing, and wherein the flush port is fluidly coupled to the main lumen by a flush lumen that extends through an adaptor spine of the handle.

Example 72. The delivery apparatus of any example herein, particularly either example 70 or 71, wherein the reservoir comprises a barrel and a movable wall disposed within the barrel, the barrel and the movable wall defining an internal cavity which contains the fluid therein, and wherein the movable wall is configured to move inward as fluid within the reservoir is transferred to the main lumen and the fluid volume decreases responsive to the fluid pressure in the main lumen being negative.

Example 73. The delivery apparatus of any example herein, particularly any one of claims 57-72, wherein the fluid inside the reservoir is saline.

Example 74. A guide sheath comprising: a handle comprising: a housing; and a flush port coupled to the housing; a reservoir fluidly coupled to the flush port and disposed external to the housing, wherein the reservoir is filled with fluid and has an adjustable volume; and a shaft extending within and distally from the handle and having a main lumen that extends within the housing, wherein the main lumen is fluidly coupled to the reservoir by the flush port, and wherein the reservoir is configured to passively supply fluid to the main lumen based on a fluid pressure in the main lumen.

Example 75. The guide sheath of any example herein, particularly example 74, wherein the handle further comprises a seal housing assembly including one or more fluid seals.

Example 76. The guide sheath of any example herein, particularly example 75, wherein the shaft extends through the seal housing assembly.

Example 77. The guide sheath of any example herein, particularly either example 75 or 76, wherein the flush port is coupled to the housing distal to the seal housing assembly.

Example 78. The guide sheath of any example herein, particularly any one of examples 74-77, wherein the reservoir comprises a barrel and a movable wall disposed within the barrel, the barrel and the movable wall defining an internal cavity which contains the fluid therein.

Example 79. The guide sheath of any example herein, particularly example 78, wherein the movable wall is configured to move inward, toward the flush port, as fluid is pulled passively from the reservoir into the main lumen and the volume of fluid in the reservoir decreases responsive to a negative pressure in the main lumen.

Example 80. The guide sheath of any example herein, particularly any one of examples 74-79, wherein the reservoir is a syringe.

Example 81. The guide sheath of any example herein, particularly any one of examples 74-80, further comprising a stopcock disposed between the flush port and the reservoir, wherein the stopcock is movable between a closed position and an open position where the reservoir is fluidly coupled to the main lumen and fluid can freely flow from the reservoir to the flush port and into the main lumen.

Example 82. The guide sheath of any example herein, particularly any one of examples 74-81, wherein the fluid inside the reservoir is saline.

Example 83. A method for implanting a prosthetic medical device, comprising: inserting a shaft of a guide catheter into a vessel of a patient, the shaft having a main lumen and extending from within and distal to a handle of the guide catheter; and inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter, wherein, as the distal end portion of the first implant catheter is advanced through the main lumen, fluid is pulled passively from a fluid reservoir into the main lumen such that a volume of the fluid reservoir decreases.

Example 84. The method of any example herein, particularly example 83, wherein fluid is pulled passively from within the fluid reservoir into the main lumen by decreasing fluid pressure within the main lumen as the distal end portion of the first implant catheter is advanced further along the main lumen.

Example 85. The method of any example herein, particularly either example 83 or 84, wherein the handle of the guide catheter includes a flush port coupled to a housing of the handle and disposed distal to one or more fluid seals of the handle that are disposed adjacent to a proximal end of the guide catheter, and wherein the fluid reservoir is fluidly coupled to the flush port.

Example 86. The method of any example herein, particularly example 85, wherein the fluid reservoir is disposed external to the handle and is coupled to the flush port, and wherein the flush port is fluidly coupled to the main lumen by a flush lumen that extends through the handle.

Example 87. The method of any example herein, particularly any one of examples 83-85, wherein the fluid reservoir is disposed within the handle, and wherein the fluid reservoir is fluidly coupled to the main lumen by a channel disposed within the handle.

Example 88. The method of any example herein, particularly example 87, wherein the fluid reservoir is disposed around the shaft, and wherein as fluid is pulled from the fluid reservoir into the main lumen, a wall of the fluid reservoir contracts inward toward the main lumen of the shaft and a fluid cavity containing the fluid and defined by the wall of the fluid reservoir decreases in size.

Example 89. The method of any example herein, particularly any one of examples 83-88, further comprising implanting the prosthetic medical device at the target implantation site, removing the first implant catheter from the guide catheter, and inserting a second implant catheter into the guide catheter and advancing the second implant catheter through the main lumen toward the target implantation site.

Example 90. The method of any example herein, particularly example 89, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 91. The method of any example herein, particularly any one of examples 43-56 or 83-90, wherein the method is performed on a living animal or on a non-living simulation.

Example 92. A method comprising sterilizing the prosthetic heart valve, apparatus, and/or assembly of any example.

Example 93. A prosthetic heart valve of any one of examples 1-92, wherein the prosthetic heart valve is sterilized.

The features described herein with regard to any example can be combined with other features described in any one or more of the other examples, unless otherwise stated. For example, any one or more of the features of one guide catheter can be combined with any one or more features of another guide catheter. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a handle;
a shaft extending within and distally from the handle and having a main lumen; and
a reservoir fluidly coupled to the main lumen, wherein the reservoir is filled with fluid and has an adjustable fluid volume, and wherein the reservoir is configured to passively supply fluid to the main lumen based on a fluid pressure in the main lumen.

2. The delivery apparatus of claim 1, wherein the handle further comprises a flush port coupled to a housing of the handle, and wherein the flush port is fluidly coupled to the reservoir.

3. The delivery apparatus of claim 2, wherein the handle further comprises a seal housing assembly including one or more fluid seals, and wherein the flush port is disposed distal and adjacent to the seal housing assembly.

4. The delivery apparatus of claim 1, wherein the main lumen is fluidly coupled to the reservoir by a first channel disposed within the handle, and wherein the reservoir comprises a movable wall that is configured to conform to the adjustable fluid volume and move inward as fluid within the reservoir is transferred to the main lumen via the first channel and the fluid volume decreases.

5. The delivery apparatus of claim 1, wherein the handle comprises a housing and the reservoir is disposed internal to the housing.

6. The delivery apparatus of claim 5, wherein a wall of the reservoir comprises a flexible material, and wherein the reservoir is configured to compress as its fluid volume reduces.

7. The delivery apparatus of claim 5, wherein the handle further comprises a flush port coupled to a housing of the handle, and wherein a second channel extends from the flush port and radially inward into an interior cavity of the reservoir.

8. The delivery apparatus of claim 5, wherein the reservoir is annular and extends axially along a portion of the handle, and wherein the reservoir is disposed radially outward of the shaft, relative to a central longitudinal axis of the delivery apparatus.

9. The delivery apparatus of claim 1, wherein the handle comprises a housing and the reservoir is disposed external to the housing.

10. The delivery apparatus of claim 9, wherein the reservoir is fluidly coupled to a flush port that is coupled to the housing, and wherein the flush port is fluidly coupled to the main lumen by a flush lumen that extends through an adaptor spine of the handle.

11. The delivery apparatus of claim 9, wherein the reservoir comprises a barrel and a movable wall disposed within the barrel, the barrel and the movable wall defining an internal cavity which contains the fluid therein, and wherein the movable wall is configured to move inward as fluid within the reservoir is transferred to the main lumen and the fluid volume decreases responsive to the fluid pressure in the main lumen being negative.

12. A guide sheath comprising:
a handle comprising:
a housing; and
a flush port coupled to the housing;
a reservoir fluidly coupled to the flush port and disposed external to the housing, wherein the reservoir is filled with fluid and has an adjustable volume; and
a shaft extending within and distally from the handle and having a main lumen that extends within the housing, wherein the main lumen is fluidly coupled to the reservoir by the flush port, and wherein the reservoir is configured to passively supply fluid to the main lumen based on a fluid pressure in the main lumen.

13. The guide sheath of claim 12, wherein the handle further comprises a seal housing assembly including one or more fluid seals.

14. The guide sheath of claim 13, wherein the shaft extends through the seal housing assembly.

15. The guide sheath of claim 13, wherein the flush port is coupled to the housing distal to the seal housing assembly.

16. The guide sheath of claim 12, wherein the reservoir comprises a barrel and a movable wall disposed within the barrel, the barrel and the movable wall defining an internal cavity which contains the fluid therein.

17. The guide sheath of claim 16, wherein the movable wall is configured to move inward, toward the flush port, as fluid is pulled passively from the reservoir into the main lumen and the volume of fluid in the reservoir decreases responsive to a negative pressure in the main lumen.

18. A method for implanting a prosthetic medical device, comprising:
inserting a shaft of a guide catheter into a vessel of a patient, the shaft having a main lumen and extending from within and distal to a handle of the guide catheter; and
inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter,
wherein, as the distal end portion of the first implant catheter is advanced through the main lumen, fluid is pulled passively from a fluid reservoir into the main lumen such that a volume of the fluid reservoir decreases.

19. The method of claim 18, wherein fluid is pulled passively from within the fluid reservoir into the main lumen by decreasing fluid pressure within the main lumen as the distal end portion of the first implant catheter is advanced further along the main lumen.

20. The method of claim 18, wherein the handle of the guide catheter includes a flush port coupled to a housing of the handle and disposed distal to one or more fluid seals of the handle that are disposed adjacent to a proximal end of the guide catheter, and wherein the fluid reservoir is fluidly coupled to the flush port.
